(19) United States
(12) Patent Application Publication
(10) Pub. No.: US 2008/0166776 A1
(43) Pub. Date: Jul. 10, 2008

(54) Method for Preparing a Useful Secondary Metabolite by Effective Elimination of Biological By-Products

(75) Inventors: Sang-Jun Sim, Seoul (KR); Sang-Woo Park, Gyeonggi-do (KR); Sung-Tae Yoon, Seoul (KR)

Correspondence Address: INTELLECTUAL PROPERTY / TECHNOLOGY LAW, PO BOX 14329, RESEARCH TRIANGLE PARK, NC, 27709, US

(73) Assignee: Sungkyunkwan University Foundation Corporate Collaboration, Suwon-si, Gyeonggi-do (KR)

(21) Appl. No.: 11/817,022

(22) PCT Filed: Mar. 16, 2006
(86) PCT No.: PCT/KR2006/000926

§ 371 Date: Sep. 19, 2007

(30) Foreign Application Priority Data
Mar. 15, 2006 (KR) 10-2005-0021421

(51) Int. Cl. C12P 17/16

(52) U.S. Cl. 435/118

(57) ABSTRACT

The present invention relates to a method that improves productivity of a useful secondary metabolite by eliminating biological by-products effectively during the culture of a useful secondary metabolite-producing microorganism, and more particularly, in order to efficiently eliminate biological by-products formed during the culture of Sorangium cellulosum, the present invention relates to a method for producing epothilones comprising that a cation exchange resin is added to said culture medium or said culture broth is recirculated through a column charged with a cation exchange resin. According to the present invention, the effective elimination of by-products formed during the culture of Sorangium cellulosum enhances growth rate of the microorganism as well as dramatically improves productivity of epothilones, a useful secondary metabolite

### TECHNICAL FIELD

The present invention relates to a method for producing a useful secondary metabolite with high yields by effective elimination of biological by-products, and more particularly, relates to a method for producing epothilones that a cation exchange resin is added to culture medium of Sorangium cellulosum or the culture broth of Sorangium cellulosum is recirculated through a column charged with a cation exchange resin in order to eliminate biological by-products formed during the culture of Sorangium cellulosum.

### BACKGROUND ART

The epothilones are antifungal and cytotoxic compounds produced by the myxobacteria, Sorangium cellulosum (Gerth, K. et al., J Antibiotics, 49:560, 1996; Bollag, D. et al., Cancer Res., 55:2325, 1995). At present, the epothilones are known to suppress proliferation of cancer cells by inhibiting cell division and stabilizing microtubules like taxol and taxotel widely used as anticancer agents. Especially, the epothilones have strong therapeutic potential against cancer cells resistant to taxol derivatives and other anticancer drugs. Therefore, this characteristic is considered as a practical solution to overcome drug resistance which is a great obstacle of cancer therapy.

The epothilones featuring an superb anticancer effect have been disclosed in a method for isolating and purifying epothilones (WO 02/46196A1), a recombinant method for producing epothilones and epothilone derivatives (WO 00/31247A2), microbial transformation for epothilone production (WO 00/3927A2), administration method of epothilone variants for cancer therapy (WO 02/58700A1) and the like.

The epothilones produced by Sorangium celluosum, a myxobacterium living in the soil have a notable antitumor effect but a tiny quantity of production hinders their wide-spread uses. Chemical synthesis of epothilones was tried to overcome the limit but proved inefficient due to their high cost and time-consuming process.

As one of the methods producing epothilones with high yields, the next generation antitumor agents, a method for producing epothilones and their derivatives using recombinant microorganism transformed by an epothilone-synthetic gene (WO 00/31247A2) is known but the process still appears unsatisfying due to low yield and antibiotics supplementation during the culture process.

### SUMMARY OF THE INVENTION

The present inventors have made extensive efforts to overcome low productivity of epothilones from natural strain, Sorangium cellulosum and found that the yield of epothilones could be remarkably augmented by culturing the microorganism while eliminating biological by-products formed during the culture of the microorganism, thereby completing the present invention.

Therefore, the main object of the present invention is to provide a method for producing epothilones with high yields by effective elimination of biological by-products formed during the culture of an epothilone-producing microorganism.

To achieve the object above, the present invention provides a method for preparing epothilones, the method comprising the steps of: (a) culturing the epothilone-producing producing microorganism while adding a cation exchange resin to the culture medium of the epothilone-producing microorganism or recirculating the culture broth of the epothilone-producing microorganism through a column charged with a cation exchange resin, in order to eliminate by-products formed during the culture of the epothilone-producing microorganism; and (b) retrieving epothilones from the culture broth.

In the present invention, the step (a) can be characterized by culturing the epothilone-producing microorganism while adding a cation exchange resin to said culture medium and simultaneously recirculating said culture broth through the column charged with a cation exchange resin.

The present invention also provides a method for preparing a useful secondary metabolite, the method comprises: (a) culturing a useful secondary metabolite-producing microorganism while adding a cation exchange resin to the culture medium of the useful secondary metabolite-producing microorganism and simultaneously recirculating the culture broth of the useful secondary metabolite-producing microorganism through a column charged with a cation exchange resin, in order to eliminate by-products formed during the culture of the useful secondary metabolite-producing microorganism; and (b) retrieving the useful secondary metabolite from the culture broth.

Other features and examples of the invention are clarified by the minute descriptions and attached claims as follows.

### BRIEF DESCRIPTION OF DRAWINGS

FIG. 1 shows the concentration of ammonium residues, a typical biological metabolite, according to the use of a selective cation exchange resin in batch culture.

FIG. 2 illustrates the immobilization process of Sorangium cellulosum.

FIG. 3 shows the starch consuming rate according to alginate concentration.

FIG. 4 shows the concentration of epothilone A and B produced by immobilized Sorangium cellulosum in continuous culture.

FIG. 5 illustrates a continuous culture mode of immobilized Sorangium cellulosum using a cation exchange resin.

FIG. 6 shows the concentration of epothilone A and B produced by immobilized Sorangium cellulosum using a cation exchange resin in continuous culture.

### DETAILED DESCRIPTION OF THE INVENTION, AND PREFERRED EMBODIMENTS THEREOF

The present invention relates to a method for producing epothilones with high yield by efficiently eliminating by-products formed during the culture of an epothilone-producing microorganism, in the process for preparing epothilones by culturing the epothilone-producing microorganism.

In the present invention, in order to effectively eliminate by-products, the culture was carried out while adding a cation exchange resin to the culture medium of the epothilone-producing microorganism or recirculating the culture broth of the epothilone-producing microorganism through a column charged with a cation exchange resin. Further, in order to effectively eliminate the by-products, the culture was carried out while adding a cation exchange resin to the culture medium of the epothilone-producing microorganism and simultaneously recirculating the culture broth of the epothilone-producing microorganism through a column charged with a cation exchange resin.

In the present invention, the epothilone-producing microorganism is preferably Sorangium cellulosum. However, it is not limited thereto; the genus Myxococcus, the genus Pseudomonas, the genus Streptomyces having the ability to produce epothilones and a microorganism transformed by epothilone-synthetic genes can be also used. Moreover, the microorganism is preferably immobilized; in this case, the immobilized microorganism can be reused.

In the present invention, said cation exchange resin is preferably resins such as Amberite FPC 22 Na and Amberite 1200C Na that do not bond with epothilones but it is not limited thereto. Also, it is preferable to supplement an absorption resin additionally to the culture medium in order to efficiently eliminate by-products, and the absorption resin is preferably XAD-16 but it is not limited thereto. Furthermore, said column is additionally charged with an absorption resin as well as a cation exchange resin.

In a preferred embodiment of the present invention, Amberite FPC 22 Na or Amberite 1200C Na as a cation exchange resin, and XAD-16 as an absorption resin were used during the culture of Sorangium cellolosum DSM 6773. In the case of adding (XAD-16+Amberite FPC 22 Na) and (XAD-16+Amberite 1200c Na) to the culture medium, biological by-products (ammonium), derived from Sorangium cellulosum DSM 6773, were eliminated by up to about 90% and epothilone productivity remarkably increased.

The method according to the present invention is not restricted to the production of epothilones. That is, in the process of preparing a useful secondary metabolite by culturing a microorganism, the yield of the useful secondary metabolite can be elevated by the efficient elimination of by-products which is adding a cation exchange resin to culture medium and simultaneously recirculating the culture broth through a column charged with a cation exchange resin.

A useful secondary metabolite (epothilones) produced by culturing a useful secondary metabolite (epothilones)-producing microorganism can be retrieved from culture broth using the conventional method. For example, a method in which cells are removed from the culture broth by centrifugation, etc, and then, the culture broth, from which cells have been removed, is applied to chromatography, etc, to retrieve a useful secondary metabolite (epothilones)

### EXAMPLES

Hereinafter, the present invention will be described in more details by examples. However, it is obvious to a person skilled in the art that these examples are for illustrative purpose only and are not construed to limit the scope of the present invention.

Especially, the following examples only illustrate the production of epothilones as a secondary metabolite. However, it is obvious to a person skilled in the art that applying the inventive method to any methods for producing other useful secondary metabolites can improve productivity of other useful secondary metabolites by effectively eliminating by-products thereof.

Besides, the following examples only illustrate Sorangium cellulosum as an epothilone-producing microorganism. However, it is not limited thereto; the genus Myzococcus, the genus Psudomonas, the genus Streaptomyces having the ability to produce epothilones and a microorganism transformed by epothilone-synthetic genes may also be used.

### Example 1

### Elimination of Biological By-Products in Suspended Culture

In order to effectively eliminate biological by-products formed from Sorangium cellulosum DSM 6773, three kinds of cation exchange resins were selected. In order to estimate the production amount of epothilones and the elimination rate of ammonium, a biological by-product by each selected cation exchange resin, cation exchange resins such as 8 g/L Duolite A7, Abmerite FPC 22 Na and Amberite 1200C Na (Rohm and Haas Co., USA) were respectively mixed with 20 g/L XAD-16 (Rohm and Haas Co., USA), and then the mixtures were added to culture medium to incubate at 32° C. and 200 rpm.

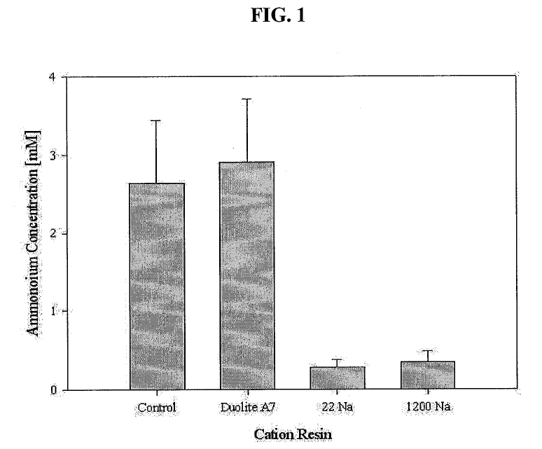

As a result, as shown in FIG. 1, 22 Na (a group added with XAD-16+amberite FPC 22 Na) and 1200 Na (a group added with XAD-16+Amberite 1200C Na) resulted in about 90% elimination rate of the biological by-product (ammonium) formed from Sorangium cellulosum DSM 6773. Also, as shown in Table 1, a group added with [XAD-16+Amberite FPC 22 Na] and a group added with [XAD-16+Amberite 1200C Na] resulted in increasing amount of epothilone production compared to a group added with only [XAD-16].

Meanwhile, each selected cation exchange resin above was added to culture medium in order to determine the possible interaction with epothilones. As shown in Table 1, it was confirmed that no interaction occurred between epothilones and the selected cation exchange resins.

TABLE 1

| | Total average epothilones |
|---|---|
| Resin | (mg/L) |
| 20 g/L XAD-16 | 2.51 |
| 20 g/L XAD-16 + 8 g/L Duolite A7 | 2.68 |
| 20 g/L XAD-16 + 8 g/L Amberite FPC 22 Na | 5.08 |
| 20 g/L XAD-16 + 8 g/L Amberite 1200C Na | 3.31 |
| 8 g/L Duolite A7 | ND |
| 8 g/L Amberite FPC 22 Na | ND |
| 8 g/L Amberite 1200C Na | ND |

ND: Not detectable

### Example 2

### Immobilization of Sorangium cellulosum DSM 6773 Using Alginate

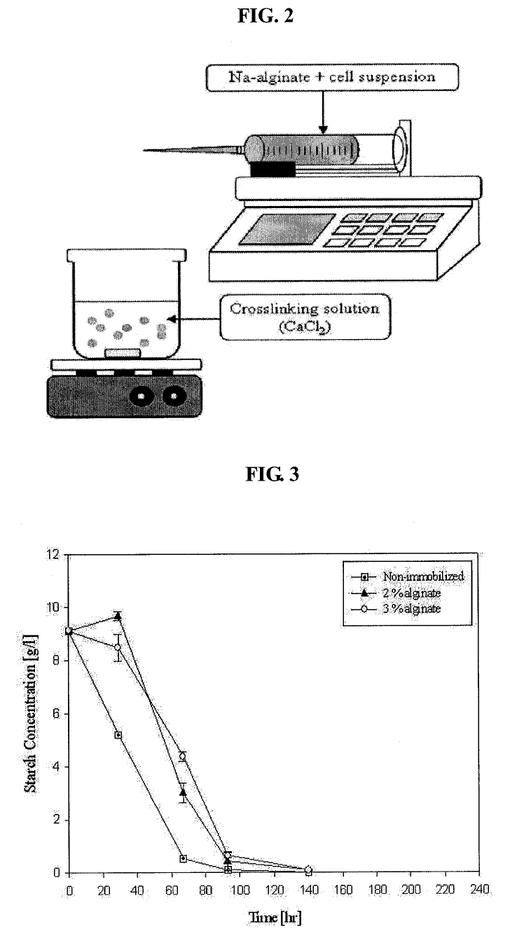

Cell growth and epothilone productivity affected by the immobilization of Sorangium cellulosum DSM 6773 were estimated by the following procedure; After 2˜3% sodium alginate was mixed with the solution of 0.9% sodium chloride, the mixture was added with cultured Sorangium cellulosum DSM 6773 at the 5:1 ratio, the suspension of which was injected into the solution of 3% calcium chloride using sylinger to immobilize the microorganism (FIG. 2). The immobilized Sorangium cellulosum DSM 6773 was incubated in the same condition as in Example 1. The consuming rate of starch, the main nutrient, was analyzed to examine the growth of Sorangium cellulosum DSM 6773 indirectly.

As a result, as in FIG. 3, the immobilization rate decreased when Sorangium cellulosum DSM 6773 was immobilized with 2% sodium alginate. Therefore, Sorangium cellulosum DSM 6773 was immobilized with 3% sodium alginate for the ensuing experiment.

While Sorangium cellulosum DSM 6773 immobilized with 3% alginate was cultured, the retrieval of epothilones released out of the cells was achieved by 20 g/L XAD-16 wrapped in gauze and added together with nutrients. The immobilized microorganism above was reused four times to produce epothilones. The first culture period was 7 days, and the second, third and fourth period were 5 days respectively.

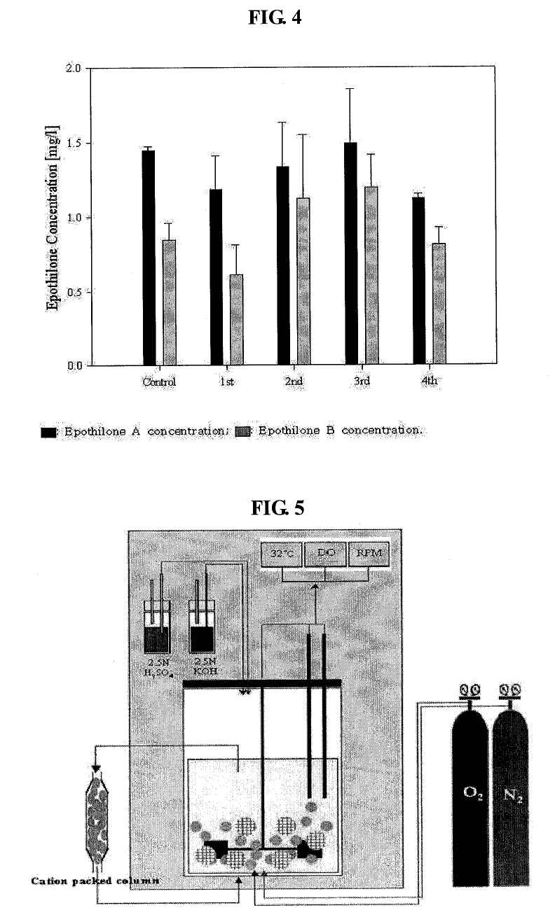

As a result, as shown in FIG. 4 it was confirmed that the immobilized Sorangium cellulosum DSM 6773 could be reused more than 4 times. Time saving for the culture and increasing productivity of epothilone A and B could be achieved by continuous culture using an immobilization system.

### Example 3

### Continuous Culture of Immobilized Sorangium cellulosum DSM 6773 Using a Cation Exchange Resin

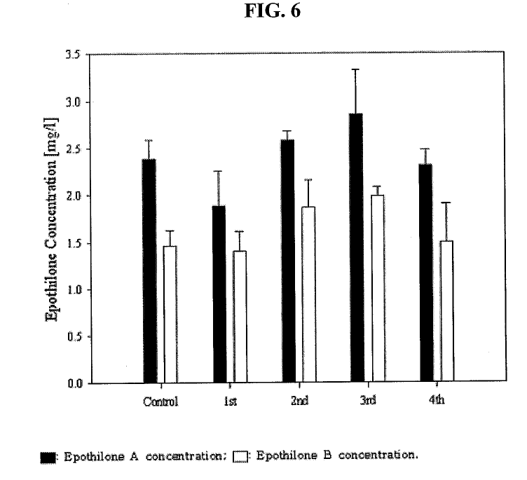

As in Example 1, Sorangium cellulosum DSM 6773 immobilized as in Example 2 was cultured while the culture broth was recirculated through a column charged with 8 g/L Amberite FPC 22 Na and 20 g/L XD-16, followed by adding 8 g/L Amberite FPC 22 Na (a cation exchange resin) and 20 g/L XAD-16 (an absorption resin) wrapped in gauze to the culture medium (FIG. 5). As a result, as shown in FIG. 6, it was confirmed that culture time was saved and productivity of epothilone A and B increased. That is, productivity of epothilone A and B was about 0.549 mg/day in suspended culture, whereas about 0.744 mg/day in the present example based on the culture time.

### INDUSTRIAL APPLICABILITY

As described in detail above, the present invention enhances the growth rate of Sorangium cellulosum by means of efficient elimination of biological by-products formed during the culture of Sorangium cellulosum as well as dramatically improves productivity of epothilones, a useful secondary metabolite. Therefore, the present invention is useful to produce epothilones cost-effectively.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A method for preparing epothilones, the method comprising the steps of:
(a) culturing an epothilone-producing microorganism while adding a cation exchange resin to a culture medium of the epothilone-producing microorganism and/or recirculating the culture broth of the epothilone-producing microorganism through a column charged with a cation exchange resin, in order to eliminate by-products produced during the culture of said epothilone-producing microorganism; and
(b) retrieving the epothilones from the culture broth.

2. The method for preparing epothilones according to claim 1, wherein the step (a) is culturing the epothilone-producing microorganism while adding a cation exchange resin to culture medium and simultaneously recirculating the culture broth through the column charged with a cation exchange resin.

3. The method for preparing epothilones according to claim 1, wherein an epothilone-producing microorganism is Sorangium cellulosum.

4. The method for preparing epothilones according to claim 1, wherein said cation exchange resin does not interact with epothilones.

5. The method for preparing epothilones according to claim 4, wherein said cation exchange resin is Amberite FPC 22 Na or Amberite 1200C Na.

6. The method for preparing epothilones according to claim 1, wherein said culture medium is additionally supplemented with an absorption resin.

7. The method for preparing epothilones according to claim 1, wherein said column is charged with a cation exchange resin and an absorption resin.

8. The method for preparing epothilones according to claim 6, wherein an absorption resin is XAD-16.

9. The method for preparing epothilones according to claim 1, wherein said microorganism is immobilized.

10. The method for preparing epothilones according to claim 9, wherein said immobilized microorganism is reused.

11. A method for preparing a useful secondary metabolite, the method comprises:
(a) culturing a useful secondary metabolite-producing microorganism while adding a cation exchange resin to culture medium of the useful secondary metabolite-producing microorganism and simultaneously recirculating the culture broth of the useful secondary metabolite-producing microorganism through a column charged with a cation exchange resin, in order to eliminate by-products formed during the culture of the useful secondary metabolite-producing microorganism; and
(b) retrieving the useful secondary metabolite from the culture broth.

12. The method according to claim 11, wherein said culture medium is additionally supplemented with an absorption resin.

13. The method according to claim 11, wherein said column is charged with a cation exchange resin and an absorption resin.

14. The method according to claim 11, wherein said microorganism is immobilized.

15. The method for preparing epothilones according to claim 2, wherein an epothilone-producing microorganism is Sorangium cellulosum.

16. The method for preparing epothilones according to claim 2, wherein said cation exchange resin does not interact with epothilones.

17. The method for preparing epothilones according to claim 7, wherein an absorption resin is XAD-16.

18. The method for preparing epothilones according to claim 2, wherein said microorganism is immobilized.
